(19) United States
(12) Patent Application Publication
(10) Pub. No.: US 2008/0082032 A1
(43) Pub. Date: Apr. 3, 2008

(54) Wrist splint allowing freedom of motion for fingers and thumb

(75) Inventor: Roy A. Meals, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Roy A. Meals, M.D., Los Angeles, CA (US)

(21) Appl. No.: 11/899,764

(22) Filed: Sep. 7, 2007

Related U.S. Application Data
(60) Provisional application No. 60/827,593, filed on Sep. 29, 2006.

(51) Int. Cl. A61F 5/00

(52) U.S. Cl. 602/21

(57) ABSTRACT

A wrist splint is formed from two rods, preferably parallel and substantially straight, with straps or bands at one end to secure the rods to the wrist and forearm area of the user such that the rods extend over the back (dorsal) side of the wrist and hand. The ends of the rods opposite the straps or bands are bent back along the lengths of the rods and are spaced apart to pass between the middle finger and the adjacent fingers of the wearer with one on each side of the middle finger, and to continue toward the mid-palm. The splint maintains the wrist in a flat, extended position without any fabric covering, or in contact with, the hand of the wearer, and with no restriction on the movement of the thumb or of any of the fingers.

### CROSS-REFERENCE TO RELATED APPLICATION

This application claims benefit from U.S. Provisional Patent Application No. 60/827,593, filed Sep. 29, 2006, the contents of which are incorporated herein by reference in their entirety.

### BACKGROUND OF THE INVENTION

1. Field of the Invention

This invention lies in the field of medical devices for the treatment of injuries to the wrist and adjacent areas.

2. Description of the Prior Art

Wrist splints are used for treatment of a wide variety of conditions. One example is carpal tunnel syndrome, a condition that is caused by the compression of a nerve crossing the wrist, and is accompanied by numbness and tingling in the fingers. The first step in the treatment of carpal tunnel syndrome is night splinting to prevent pressure on the nerve that is caused by sleeping in a curled-up, fetal position with the wrist in prolonged flexion. The second step is daytime splinting to preclude pressure on the nerve by awkward wrist positioning as commonly encountered by the use of a computer keyboard or while driving a car. Wrist splinting is also used for treating wrist sprains, which arise from stretching the ligamentous connecting tissues holding the wrist bones to the forearm bones and are often caused by falling on an outstretched hand. The resolution of wrist sprains typically requires weeks or months of limiting the activity and motion of the wrist, and hence a splint.

Wrist splints are also used to facilitate healing after a wide variety of surgical procedures performed on the forearm or wrist. These procedures include fracture fixation in the hand, wrist and forearm, wrist fusion, tendon repairs and transfers, wrist arthroscopy, and joint replacement. At the completion of surgery, the surgeon typically immobilizes the wrist by applying a gauze surgical bandage and a plaster of paris slab overwrapped with a cotton roller bandage. This forms a soft bandage with a rigid strut, the gauze surgical bandage being both absorptive of blood and expandable to accommodate swelling. When sutures are applied, the sutures are typically removed ten days after surgery, and once this is done, the surgeon often applies a rigid circumferential cast of plaster of paris or fiberglass if full-time, enforced wrist immobilization is required. When the cast is removed, it is often replaced by a wrist splint. In certain treatments, the cast is eliminated entirely and a wrist splint is applied directly after suture removal. In either case, the splint is designed to limit wrist motion and yet be removable by the patient to allow the patient to bathe and perform rehabilitation exercises.

Wrist splints are also used for nonoperative management of a variety of forearm, wrist and hand conditions where a moderate degree of wrist immobilization is desired, sometimes as much for comfort as for protection. Examples of these conditions are tendonitis, bruises, minor fractures, and tennis elbow. Unlike a cast, a splint offers the advantage of being is removable for ease of wound care, for dressing and undressing, for bathing, for rehabilitation exercises, and for allowing the individual to function effectively in social settings or in business activities.

Wrist splints of the prior art use fabric to encircle the palm, the back of the hand, and the distal portion of the forearm. The fabric is typically formed as a cylinder with a side hole for the thumb. The distal portion of the hand, i.e., the index, middle, ring, and small fingers, protrudes from one end of the hollow cylinder while the mid-forearm protrudes from the other end. Splint material adjacent to the thumb hole passes from the palm through the region between the index finger and the base of the thumb (which region is also referred to as the first web space or the thumb-index web space) to the back of the hand. In some cases, the wrist splint is formed by wrapping a panel of material around the hand and forearm to form the cylinder and then securing the overlapping or abutting ends of the panel to each other with VELCRO® (Velcro USA, Inc.) (generically referred to herein as “hook-and-loop”) fasteners. In others, the wrist splint is a pre-formed cylinder that is slipped onto the hand and forearm in an expanded condition, similar to applying a sock to one's foot, then drawn to conform to the shape of the hand and forearm and secured with VELCRO fasteners or with a shoestring passed through eyelets. In both cases, a metal or plastic strut is often included in a pocket sleeve of the splint to enhance the rigidity of the splint and to further limit wrist extension and flexion. If only one strut is present, the strut is typically on the palm side of the splint and extends from nearly one end of the cylinder to nearly the other end. Some struts are permanently sewn in while others are removable to allow the user to launder the fabric portions of the splint. Some splints have two struts, spaced apart from each other by 180 degrees around the cylinder. The added rigidity provided by the second strut enhances the immobilization effect. These factory-made splints are typically sized extra small, small, medium, large, and extra large, and are made specifically to fit either the right hand or the left hand.

The splints of the prior art suffer certain inadequacies. One is that the fabric passing from the palm to the back of the hand across the first web space restricts the movement of the thumb and chafes the underlying skin. Another is that the thumb hole is often too small or not placed in a location that will allow unimpeded movement of the thumb. Such a splint restricts digital motion and function. Another problem is that the proximal edge of the rim around the thumb hole digs into the skin at the base of the thumb at the wrist level and compresses the superficial radial nerve immediately underlying this area of the skin. This causes numbness and tingling on the backs of the thumb and index finger. A still further problem arises from the fact that the splint fabric covers the majority of the palm. This prevents the wearer from washing the hand effectively with the splint in place, and when the fabric and strut extend distally beyond the most distal of the palmar skin creases, the splint restrains the index, middle, ring, and small fingers in a straight position, thereby preventing the wearer from making a complete fist.

Still further, the fabric and strut contact the hand over the site at the “heel” of the hand where the median nerve is compressed in carpal tunnel syndrome. Additional pressure at this site can worsen the effects of the syndrome, especially when the splint is worn tightly, even when the splint is holding the wrist in the favored straight position, i.e., unbent either forward or backward. The splint also adds considerable circumferential bulk to the hand, wrist, and forearm, preventing the wearer from wearing a watch or a bracelet over the splint. The increased circumference and the irregular contours of the VELCRO straps also make it difficult for the wearer to reach into a trouser pocket, to pass the hand through a narrow sleeve, or to reach into a deep purse.

As noted above, most wrist splints are designed specifically for either a left hand or a right hand and are not interchangeable between left and right. This requires the supplier to stock both the left-hand version and the right-hand version, and thereby maintain a separate inventory for each hand. This lack of interchangeability also prevents one from using the same splint first to treat an injury on one wrist and later to treat an injury on the other wrist.

Further disadvantages are the tendency of perspiration and body oils, as well as environmental dirt and liquids, to permeate and adhere to the splint fabric, raising both sanitation concerns and aesthetic concerns. A second splint is needed for maintaining wrist protection while the first is being washed and dried. Splints of the prior art are also costly to manufacture since they require considerable sewing and shaping of the various fabric and strut materials. Finally, coins, buttons, paper clips, and similarly small objects cannot be effectively held in the palm when a splint of the prior art is in place because the fabric covers the skin and prevents the wearer from sensing the precise location of the object through the wearer's skin. When the wearer makes a fist around a small object, the object can inadvertently slip around the distal edge of the splint cylinder and lodge between the cylinder and the palm skin. To recover the object, the wearer must then shake the object out or remove the splint.

### SUMMARY OF THE INVENTION

This invention resides in a wrist splint that includes a dorsal bar and a pair of rods extending from one end of the bar, the rods spaced apart by a sufficient width to permit the passage of the wearer's middle finger between them and curved in the palmar direction to pass through the wearer's web spaces on either side of the middle finger. The rods extend a short distance over the palm, securing the rods to the hand at the base of the middle finger and thereby maintaining alignment of the palm with the forearm. In certain embodiments the dorsal bar is itself a pair of rods which are an extension of the two rods that pass through the web spaces, while in other embodiments the dorsal bar is a unitary bar to which the proximal ends of the rods are embedded or otherwise secured. In either case, the bar and rods are rigid or semi-rigid and the splint is shaped to extend from the back of the forearm and from there to pass distally over the back of the wrist and hand, then to pass between the middle finger and adjacent fingers at the base of the middle finger toward the palm, preferably with one rod on each side of the middle finger. In preferred structures, the palmar ends of the rods terminate at approximately the mid-palm. The ends of both rods at the mid-palm (i.e., the distal ends) can be joined such that the two wires form a loop. The proximal (forearm) ends of the rods are securable to the forearm by one or more double-sided VELCRO straps or their equivalents, such as elastic bands or any rigid or flexible cuffs that are secured by straps, clasps, snaps, buttons, or any other conventional fasteners. In preferred embodiments of the invention, two such cuffs or VELCRO straps are used, the one closest to the elbow encircling the forearm approximately midway between the wrist and elbow, and the other encircling the forearm at a location immediately proximal to the wrist, at the site where one would wear a wristwatch.

Among the advantages of wrist splints of this invention are that the area between the thumb and index finger remains entirely uncovered and unconstrained when the user is wearing the splint. This avoids chafing and any limitation on the motion of the thumb and index finger. Also, the splint material is far removed from the thumb, thereby preserving critical functions of the hand. Further still, the area at the wrist where the superficial radial nerve is vulnerable to compression from the proximal edge of the thumb hole in a conventional wrist splint remains entirely uncovered. Irritation of the superficial radial nerve by the splint is thus avoided. Preferred splints of this invention contain no components of other elements that limit the movement of any of the wearer's fingers to any substantial degree, and no rods, straps or other components that would pass over the wearer's webs other than the two webs on opposite sides of the middle finger.

Wrist splints of this invention do not place any fabric in contact with the hand itself. With a splint in place, therefore, the entire hand can be washed thoroughly with the same efficiency and effectiveness as if one were wearing a wristwatch. The wearer is also fully capable of forming a fist while the wrist splint is in place. Also, no portion of the wrist splint contacts the hand at the region of the carpal tunnel. There is thus no danger of tight application of the splint contributing to carpal tunnel syndrome. At the distal hand, the splint in the preferred embodiments of this invention adds only the thickness of four rods to the cross-sectional bulk of the hand. In the region extending from mid-hand to mid-forearm, the only thickness added by the splint to the cross-section of the region, other than that of the two VELCRO straps, is the thickness of two rods and any covering pad that may be present (see the discussion of preferred embodiments below. The two VELCRO straps themselves add no more bulk than that of a wristwatch or a bracelet. With so little additional bulk, the wearer can wear a wristwatch, or button the cuff of a long-sleeved shirt, easily over the splint, and because of the splint's extremely low profile, the splint does not interfere with the wearer's ability to slip the hand into a sleeve, a trouser pocket, or a deep purse. Under a long-sleeved shirt, only the two rods on the dorsum of the hand are visible, making the splint visibly unobtrusive. The wearer can also shake hands without the splint being noticed. Furthermore, the splint fits both left and right hands equally. The hand portions of the splint that are in ready exposure to, and contact with, the environment are entirely impervious to moisture, odors, and grime, and small objects can be held in the fist without getting lost or caught. In terms of cost, the materials and labor required for manufacture of the splint are minimal.

### BRIEF DESCRIPTION OF THE DRAWINGS

FIG. 1 is a perspective view of one example of a wrist splint in accordance with the present invention.

FIG. 2 is a palm-side view of a hand and wrist with the wrist splint of FIG. 1 mounted thereto.

FIG. 3 is a perspective view of a second example of a wrist splint in accordance with the present invention.

### DESCRIPTION OF PREFERRED EMBODIMENTS

The terms “dorsal” and “palmar” are used herein in the manner in which these terms are used in the art to refer to directions relative to the hand of the wearer when the wrist splint of this invention is deployed. “Dorsal” refers to the side of the hand opposite the palm, and to the side of the wrist and forearm that are likewise opposite the palm. “Palmar” refers to the side of the hand where the palm resides, and to the side of the wrist and forearm that are on the same side as the palm. The expression “the palmar direction” thus means the direction extending from the dorsal side of the hand to the palmar side, while “the dorsal direction” means the direction from the palmar side to the dorsal side. The terms “proximal” and “distal” are likewise used in the manner in which they are used in the medical device art. In this specification, “proximal” refers to the end of the splint that is closest to the wearer's elbow when the splint is deployed, while “distal” refers to the end furthest from the elbow. In this specification as well, the ends of the rods that are in contact with the mid-palm are also termed “distal” ends.

While the features of the wrist splint of this invention are described in reference to the hand, wrist, and forearm of the wearer, the dimensions can vary depending on the size of the wearer's hand, wrist, and forearm. Wrists splints of the invention will thus be sized for use by individuals of different sizes, which can conveniently be categorized according to the height and body build of the wearer. Appropriate dimensions for given heights and body builds will be readily apparent to those skilled in the practice of medical arts and the treatment of hand and wrist injuries.

While the features defining this invention are capable of implementation in a variety of constructions, the invention as a whole will be best understood by a detailed examination of a specific embodiment. Two such embodiments are shown in the drawings.

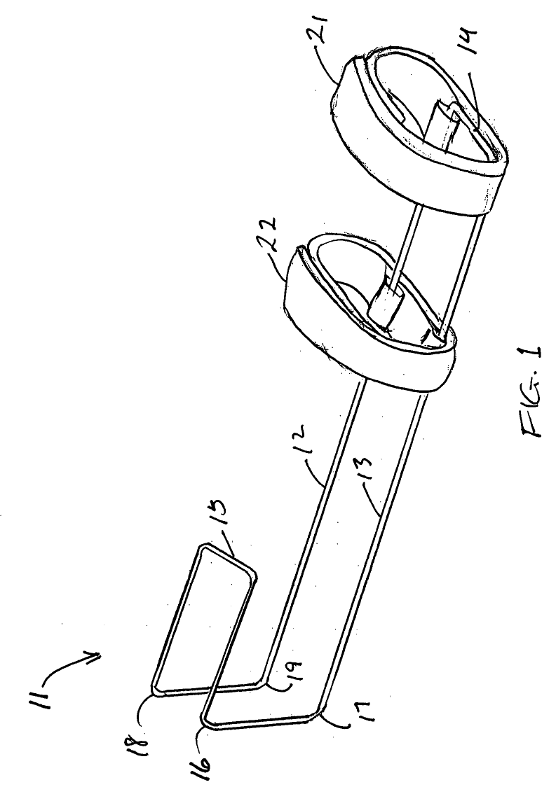

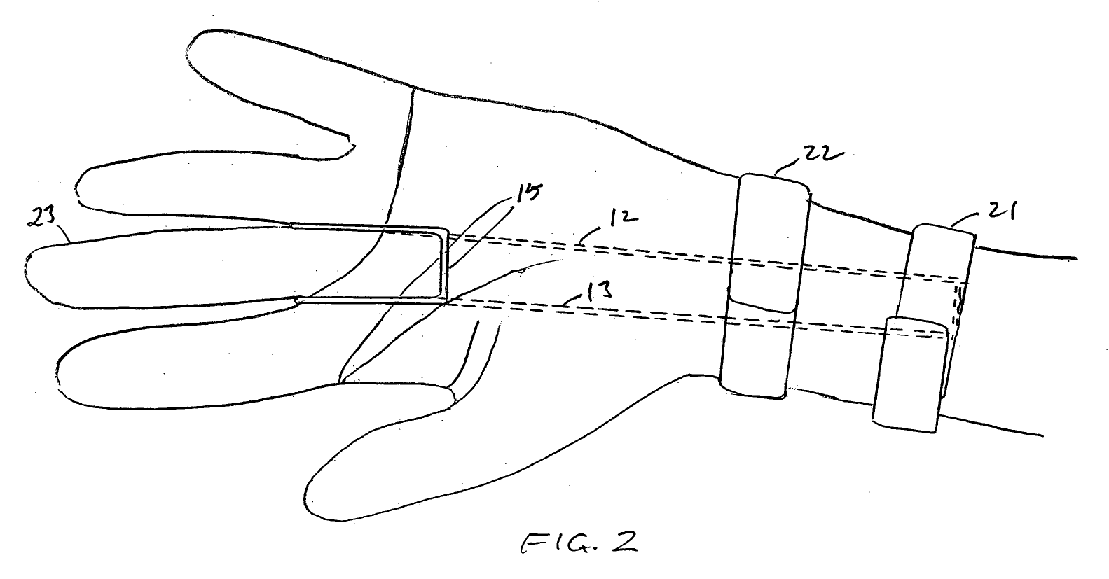

In the wrist splint 11 of FIG. 1, the dorsal bar consists of the two rods 12, 13 that are joined by a cross rod 14 at their proximal (forearm) ends and another cross rod 15 at their distal ends, i.e., the ends extending over the palm. The splint is worn with the longer portions of the rods 12, 13 running along the back of the hand, and both rods 12, 13 are bent at approximately right angles 16, 17, 18, 19 to pass from the back of the hand across the webs on either side of the middle finger to the palm. These bends can be rounded or rectangular, or any other shape that will engage the webs on either side of the middle finger. Near the proximal end of the splint are two VELCRO straps, including one strap 21 to encircle the forearm at a point approximately midway between the wrist and the elbow, and another 22 to encircle the forearm close to the wrist. FIG. 2 shows the same splint in position, with the longer portions of the rods 12, 13 in dashed lines to indicate that they are on the side of the hand that is opposite the side shown. The middle finger 23 is straddled by the two rods at the bends in the rods.

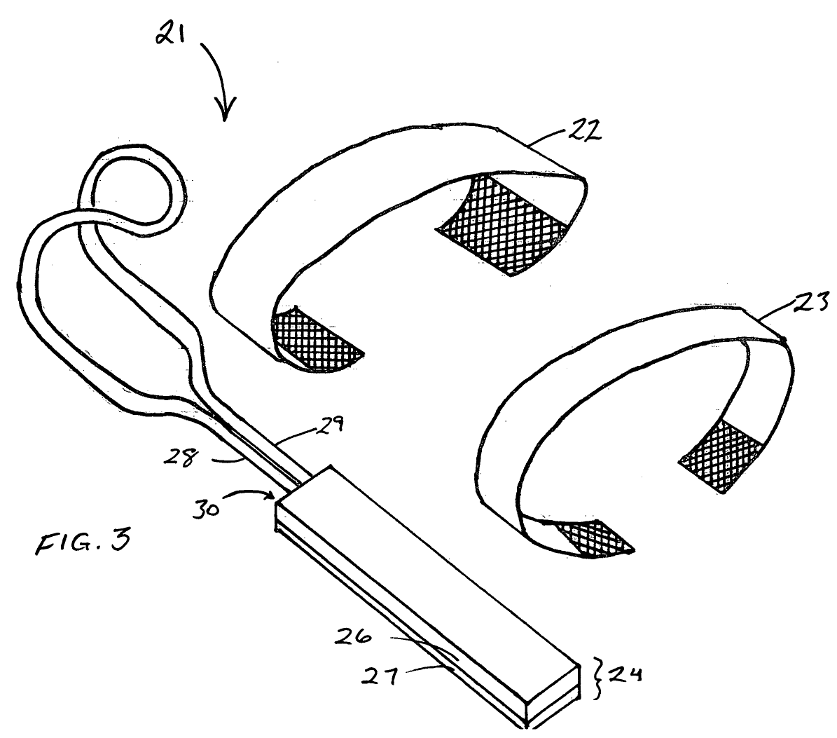

The wrist splint 21 of FIG. 3 is shown with the VELCRO straps 22, 23 separated from the remainder of the splint for ease of viewing. The dorsal bar 24 is a piece of foam 26 bonded to a VELCRO strip 27, the foam 26 providing comfort to the wearer while resting against the underside of the wearer's forearm, and the VELCRO strip 27 providing the surface features needed for securement to the VELCRO straps 22, 23. Secured between the foam 26 and the VELCRO strip 27 are two parallel rods 28, 29 that extend beyond the distal end 30 of the dorsal bar 24 where they are spread apart sufficiently to allow the passage between them of the wearer's middle finger in the same manner as the two rods 12, 13 of the wrist splint of FIGS. 1 and 2. The wrist splint 21 of FIG. 3 is worn in the same manner as the wrist splint of FIGS. 1 and 2.

The foregoing is offered primarily for purposes of illustration. Various modifications which will be readily apparent to those skilled in the art that utilize the same features of novelty and utility are all encompassed by the scope of this invention.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A wrist splint comprising:
a dorsal bar having a proximal end and a distal end,
a pair of rods extending from said distal end, said rods curved in a palmar direction and spaced apart by a distance sufficient to permit passage of said rods through adjacent web spaces on either side of a human middle finger, and
means for removably securing said dorsal bar to a forearm of a wearer.

2. The wrist splint of claim 1 wherein said rods are curved in said palmar direction to form palmar segments that terminate at a point approximately mid-palm of said wearer.

3. The wrist splint of claim 2 wherein said palmar segments of said rods are joined to form a loop.

4. The wrist splint of claim 1 wherein no portion of said wrist splint produces any substantial limitation to movement by said wearer of any finger.

5. The wrist splint of claim 1 wherein no portion of said wrist splint passes through any web space other than said web spaces on either side of said middle finger.

6. The wrist splint of claim 1 wherein said means for removably securing said dorsal bar to said forearm are comprised of a pair of straps with hook-and loop fasteners.

7. The wrist splint of claim 1 wherein said dorsal bar comprises a pair of parallel rods that are extensions of said pair of rods that are curved in said palmar direction.
